SEQUENCE LISTING (9 sequences)

SEQ ID NO: 1 - DNA, 22 bp, Artificial Sequence
  RT primer for GAPDH
cattgtcataccaggaaatgag

SEQ ID NO: 2 - DNA, 20 bp, Artificial Sequence
  RT primer for notch-delta-E
ctcgaagcattaaccctcac

SEQ ID NO: 3 - DNA, 20 bp, Artificial Sequence
  RT primer for firefly luciferase
gtgagaattcacggcgatct

SEQ ID NO: 4 - DNA, 20 bp, Artificial Sequence
  forward RT primer for GAPDH
gggtgtgaaccacgagaaat

SEQ ID NO: 5 - DNA, 20 bp, Artificial Sequence
  reverse RT primer for GAPDH
ccttccacaatgccaaagtt

SEQ ID NO: 6 - DNA, 21 bp, Artificial Sequence
  forward RT primer for notch-delta-E
agcttttgctccatagcttta

SEQ ID NO: 7 - DNA, 20 bp, Artificial Sequence
  reverse RT primer for notch-delta-E
aaggaagctaaggacctcaa

SEQ ID NO: 8 - DNA, 20 bp, Artificial Sequence
  reverse RT primer for luciferase
ggctgcaaaagatcctcaac

SEQ ID NO: 9 - DNA, 20 bp, Artificial Sequence
  forward RT primer for luciferase
aatgggaagtcacgaaggtg

(19) United States
(12) Patent Application Publication
(10) Pub. No.: US 2024/0141408 A1
(43) Pub. Date: May 2, 2024

(54) CELL-BASED ASSAY FOR IDENTIFICATION OF ACTIVATORS OF GAMMA-SECRETASE

(71) Applicant: The Brigham and Women’s Hospital, Inc., Boston, MA (US)

(72) Inventor: Jie Shen, Brookline, MA (US)

(21) Appl. No.: 18/568,612

(22) PCT Filed: Jun. 10, 2022
(86) PCT No.: PCT/US2022/032972

§ 371 Date: Dec. 8, 2023

Related U.S. Application Data
(60) Provisional application No. 63/209,121, filed on Jun. 10, 2021.

(51) Int. Cl. C12Q 1/37; C12Q 1/6851; C12Q 1/6897

(52) U.S. Cl. CPC C12Q 1/37; C12Q 1/6851; C12Q 1/6897; C12N 2503/02

(57) ABSTRACT

Described herein are cell-based high-throughput screening (HTS) assays for the identification of activators of γ-secretase.

### CLAIM OF PRIORITY

This application claims the benefit of U.S. Provisional Application Ser. No. 63/209,121, filed on Jun. 10, 2021. The entire contents of the foregoing are incorporated herein by reference.

### FEDERALLY SPONSORED RESEARCH OR DEVELOPMENT

This invention was made with Government support under Grant No. NS041783 awarded by the National Institutes of Health. The Government has certain rights in the invention.

### SEQUENCE LISTING

This application contains a Sequence Listing that has been submitted electronically as an ASCII text file named “Sequence_Listing.txt.” The ASCII text file, created on Jun. 9, 2022, is 3 kilobytes in size. The material in the ASCII text file is hereby incorporated by reference in its entirety.

### TECHNICAL FIELD

Described herein are cell-based high-throughput screening (HTS) assays for the identification of activators of γ-secretase.

### BACKGROUND

AD is the most common cause of dementia and neurodegeneration with no effective disease-modifying therapy. Dominant mutations in the PSEN1 and PSEN2 genes encoding presenilin-1 (PS1) and Presenilin2 (PS2) account for ˜90% of all familial Alzheimer's disease (FAD) mutations, highlighting the importance of Presenilin (PS) in AD pathogenesis. The clinical and neuropathological features of sporadic and familial AD are virtually indistinguishable, suggesting a common pathogenic mechanism. PS harbors the catalytic center, as an aspartic protease, of γ-secretase, which is a membrane-bound protease complex. In addition to PS, γ-secretase also includes nicastrin (NCT), APH-1, and PEN-2 as essential transmembrane cofactor proteins. γ-secretase carries out intramembranous cleavage of a subset of type I, single-span membrane proteins, including beta-amyloid precursor protein (βAPP) and Notch. Complete loss of PS or NCT function in the adult cerebral cortex of PS conditional double knockout (cDKO) mice recapitulates key features of AD, including profound age-dependent dementia, neurodegeneration, inflammatory responses and tau hyperphosphorylation. Similarly, homozygous L435F and C410Y knock in (KI) mice exhibited morphological, neurodevelopmental, and biochemical phenotypes identical to those of PSEN1 null mice. In addition, heterozygous L435F KI adult mice displayed a series of neuropathological, synaptic, and behavioral changes relevant to FAD.

### SUMMARY

Described herein are cell-based high-throughput screening (HTS) assays for the identification of activators of γ-secretase.

Thus, provided herein are isolated cells comprising: (i) a reporter gene under control of a promoter comprising at least one CBF1 binding site, and (ii) a sequence encoding NotchΔE under control of a promoter, preferably wherein the cell comprises one or more mutations in an endogenous APP, PSEN1, and/or PSEN2 gene, preferably wherein the mutations are associated with Alzheimer's disease.

In some embodiments, the promoter comprising at least one CBF1 binding site is a Hes1 proximal promoter.

In some embodiments, the sequence encoding NotchΔE is under control of a constitutive promoter. In some embodiments, the constitutive promoter is SV40, CMV, UBC, EF1A, PGK, or CAGG.

In some embodiments, the reporter gene encodes a fluorescent protein. In some embodiments, the fluorescent protein is a green fluorescent protein, a red fluorescent protein, a yellow fluorescent protein, a blue fluorescent protein, a cyan fluorescent protein, or an orange fluorescent protein.

In some embodiments, the reporter gene encodes a bioluminescent protein. In some embodiments, the bioluminescent protein is aequorin or luciferase.

In some embodiments, the isolated cell stably expresses the reporter gene and/or NotchΔE. In some embodiments, the isolated cells transiently expresses the reporter gene and/or NotchΔE.

In some embodiments, the isolated cells are mammalian cells, preferably cultured mammalian cells.

Also provide herein are methods for identifying a gamma secretase activator. The method comprise providing a cell comprising: (i) a reporter gene under control of a promoter comprising at least one CBF1 binding site, and (ii) a sequence encoding NotchΔE under control of a promoter; preferably wherein the cell comprises one or more mutations in an endogenous APP, PSEN1, and/or PSEN2 gene, and preferably wherein the mutations are associated with Alzheimer's disease; contacting the cell with a test compound; detecting a signal from the reporter gene in the presence and absence of the test compound; comparing the signal from the reporter gene in the presence and absence of the test compound; and identifying as a gamma secretase activator a compound that produces an increased signal from the reporter gene in the presence of the test compound as compared to the signal in the absence of the test compound.

In some embodiments, the promoter comprising at least one CBF1 binding site is a Hes1 proximal promoter.

In some embodiments, the sequence encoding NotchΔE is under control of a constitutive promoter.

In some embodiments, the constitutive promoter is SV40, CMV, UBC, EF1A, PGK, or CAGG.

In some embodiments, the reporter gene encodes a fluorescent protein. In some embodiments, the fluorescent protein is a green fluorescent protein, a red fluorescent protein, a yellow fluorescent protein, a blue fluorescent protein, a cyan fluorescent protein, or an orange fluorescent protein.

In some embodiments, the reporter gene encodes a bioluminescent protein. In some embodiments, the bioluminescent protein is aequorin. In some embodiments, the bioluminescent protein is luciferase, and the method further comprises lysing the cell and contacting the lysate with a luciferin substrate.

In some embodiments, the cells stably expresses the reporter gene and/or NotchΔE, or transiently expresses the reporter gene and/or NotchΔE.

In some embodiments, the cells are mammalian cells, preferably cultured mammalian cells.

In some embodiments, detecting a signal from the reporter gene comprises using imaging to detect a fluorescent or bioluminescent signal.

Also provided herein are cell-based methods to screen for compounds that activate gamma-secretase comprised of: (a) plating cells derived from animals with an FAD-linked genotype that abolishes or reduces Presenilin-1 (PS1); (b) transfecting said cells with a Notch target gene reporter system that, when activated, increases the expression of a visual or colorimetric detection system signal in said cells; (c) exposing said cells with a compound; and (d) detecting and/or quantifying a change in said visual or colorimetric detection system signal wherein (e) activation of gamma-secretase is detected by a change in the detection signal.

In some embodiments, the cells are murine embryo fibroblasts (MEFs). In some embodiments, In some embodiments, the MEF's are made immortal, e.g., by transfection with SV40. In some embodiments, the FAD-linked genotype is heterozygous for one allele that has an L435F knock-in PS1 mutation. In some embodiments, the Notch target gene reporter system transfected into said cells contains both an Hes1 proximal promoter containing two Notch responsive CBF1/TBPKappa binding sites, driving expression of firefly luciferase, and a CMV promoter cloned with NotchΔE. In some embodiments, the transfected Hes1 proximal promoter is cloned into a hygromycin resistant plasmid. In some embodiments, the CMV promoter and NotchΔE are cloned into a neomycin resistant plasmid.

In some embodiments, the detection system signal is firefly luciferase activity and is measured by luminescence. In some embodiments, wherein compounds that activate gamma-secretase are detected by an increase in the detection signal.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-E. Decreased γ-secretase activity in PS1 KI and PS1−/− cells. A, The diagram explains an exemplary embodiment in which the expression of Firefly Luciferase depends on Notch cleavage. The transmembrane protein, Notch-ΔE, is cleaved by γ-secretase releasing the Notch Intracellular Domain (NICD), which localizes to the nucleus. NICD binds to and frees the Hes1 promoter from co-repressor proteins and recruits co-activators. The activation of the Hes1 promoter allows the expression of a detectable reporter such Firefly Luciferase, which can be detected via a luminescence assay. B, Western blotting using cell lysates from immortalized MEFs derived from embryos carrying varying PS genotypes shows reduced γ-secretase activity in PS1 L435F KI and PS1−/− cells, as indicated by decreased NICD production. MEFs were transfected with 1.25 μg Hes1-Luc and 5 ng CMV-NAE. Antibodies specific for the N-terminus of PS1, cleaved notch (NICD), and α-tubulin were used. C,D, Quantification of PS1-NTF and NICD levels is shown, respectively. Protein levels are normalized to α-tubulin, and the protein levels in +/+ cells are expressed as 1. E, Luciferase activity reflects NICD cleavage. Data are the means of 4 independent experiments. All data are expressed as mean±SEM. Statistical analysis was performed using one-way ANOVA with Tukey's post-test. *p<0.05; **p<0.01, ***p<0.001 compared to +/+ cells.

FIGS. 2A-D. Exogenous wild-type human PS1 (hPS1) expression rescues decreased γ-secretase activity in PS mutant and KO MEFs. A, Western blotting using cell lysates from immortalized MEFs derived from embryos carrying varying PS genotypes. MEFs were transfected with 1.25 μg Hes1-Luc, 5 ng Notch-ΔE, and an increasing amount of plasmid expressing human PS1. Levels of NICD are indicative of γ-secretase activity, and α-tubulin is used as a control. B,C Quantification of PS1 NTF and NICD production shows increased γ-secretase activity upon transfection of wild-type hPS1 cDNA in PS1 KI and KO cells. Protein levels are normalized to α-tubulin. NICD levels in +/+MEFs is set as 1. D, The transfection of hPS1 rescues γ-secretase activity monitored via luciferase assay. Data are the means of 4 independent experiments. All data are expressed as mean±SEM.

FIGS. 3A-D. Development of cell based assay. A, The activity of luciferase is confirmed in L435F KI/+ stable cells. B, Quantitative RT-PCR analysis of luciferase using a primer pair for luciferase. mRNA expression is shown as a percentage value of the highest expressed clone. C, Quantitative RT-PCR analysis of NotchΔE using a primer pair for NotchΔE. Arrows indicate clones used for Z′ factor determination. D, The determination of Z′ factor for DMSO versus DAPT treated cells and DMSO versus the genetic positive control. Green box indicate the best clone.

FIG. 4. Testing cell based assay with γ-secretase inhibitors and modulators. Clone A23 was further validated by testing the effect of γ-secretase inhibitors (DAPT and L685,458), and modulators (Indomethacin and R-Flurbiprofen). The reduction of luciferase activity was confirmed in the γ-secretase inhibitors with lesser effect from the γ-secretase modulators.

FIG. 5. Primary screening of known bioactive and commercial library. A scatter blot of primary screening data where the Z′ score represented the number of standard deviations above the plate mean of the experimental wells. A collection of 1,902 compounds from Selleck known bioactive library and 8,098 compounds from Asinex commercial library were screened. Hits were categorized according to the Z′ score: 3-4 weak (dark blue dots), 4-5 Medium (red dots), and >5 strong (light blue).

FIGS. 6A-F. The process of Confirming and validating hits. The 3653A17 compound is used here as an example for the confirmation process. A, The compound hit from the same screening patch is confirmed in the Notch reporter assay stable cells (KI/+, KI/+;−/−, and wild-type). B, The compound hit reconstituted from a different patch is confirmed in the Notch reporter assay in a dose response experiment (4 nM to 33 μM) to calculate EC₅₀. C, Western blotting using cell lysates from KI/+ stable cells, expressing NotchΔE. Cells were treated with different doses of the compound hit. Levels of NICD are indicative of γ-secretase activity, and α-tubulin is used as a control. D, Quantification of NICD production shows increased γsecretase activity upon treatment of the hit. Protein levels are normalized to α-tubulin and uncleaved NotchΔE. NICD level in untreated cells (0 μM) is set as 1. E, The diagram explains the expression of Firefly Luciferase depending on C99 cleavage. The transmembrane protein, C99 fused to Gal4-VP16, is cleaved by γ-secretase releasing the APP Intracellular Domain (AICD) fused to Gal4-VP16, which localizes to the nucleus. The UAS promoter is activated by Gal4-VP16 allowing the expression of Firefly Luciferase, which can be detected via a luminescence assay. F, The hit is confirmed in the APP dependent secondary screen in a dose response experiment (4 nM to 33 μM).

### DETAILED DESCRIPTION

Therapeutics affecting the etiology of AD have been challenging to develop due to the multifactorial nature of the disease. Currently in the US, only acetylcholine esterase inhibitors have been approved for the management of AD without impeding disease progression. With major therapeutic efforts focusing on Aβ failing to produce an effective AD therapy, the development of new therapeutics currently stands at a crossroads. Consequently, there is an urgent need to establish unconventional strategies to develop successful therapeutics. This study provides novel disease-modifying therapeutics that are much needed as an alternative to or in combination with current symptomatic treatment.

Described herein are cell-based high-throughput screening (HTS) assays for the identification of activators of γ-secretase. Since these assays measure an increase in signal, compounds involved in cell toxicity will not be identified as false positives.

The assays make use of a Notch target gene reporter system, which takes advantage of the fact that Notch1 binds to the transcription factor CBF1 and stimulates transcription through promoters containing CBF1-responsive elements (Lu and Lux, Proc Natl Acad Sci USA. 1996 May 28;93(11):5663-7). The reporter system includes (i) a reporter gene under control of a promoter comprising at least one CBF1 binding site, e.g., a Hes1 proximal promoter containing two Notch responsive CBF1/RBPJκ binding sites, and (ii) a NotchΔE expression construct; NotchΔE is a truncated Notch construct that is readily cleaved by γ-secretase (Ray et al., Proc Natl Acad Sci USA. 1999, 96 (6): 3263-3268).

In some embodiments, the reporter gene is a gene that encodes a fluorescent protein. In some embodiments, the reporter gene encodes a green fluorescent protein, a red fluorescent protein, a yellow fluorescent protein, a blue fluorescent protein, a cyan fluorescent protein, an orange fluorescent protein, or combinations thereof.

The green fluorescent protein can be, e.g., GFP, EGFP, Emerland, Superfold GFP, Azami Green, mWasabi, TagGFP, TurboGFP, AcGFP, ZsGreen, T-Sapphire, click beetle green, and combinations thereof.

The blue fluorescent protein can be, e.g., EBFP, EBFP2, Azurite, mTagBFP, and combinations thereof.

The cyan fluorescent protein can be, e.g., ECFP, mECFP, Cerulean, mTurqoise, CyPet, AmCyan1, Midori-Ishi Cyan, TagCFP, mTFP11 (Teal), and combinations thereof.

The yellow fluorescent protein can be, e.g., EYFP, Topaz, Venus, mCitrine, YPet, TagYFP, PhiYFP, ZsYellowl, mBanana, and combinations thereof.

The orange fluorescent protein can be, e.g., Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, and combinations thereof.

The red fluorescent protein can be, e.g., mRuby, mApple, mStrawberry, AsRed2, mRFP1, JRed, mCherry, HcRed1, mRaspberry, dKeima-Tandem, HcRed-Tandem, mPlum, AQ143, or combinations thereof.

In some embodiments, the reporter protein is a luminescent protein, e.g., aequorin, or produces luminescence in the presence of a substrate, e.g., NANOLUC, firefly or jellyfish luciferase, which oxidizes luciferin to produce luminescence. See, e.g. Saito et al., Nat Commun. 2012 Dec. 11; 3: 1262. Other reporter genes that produce a colorimetric signal can also be used, e.g., horseradish peroxidase (HRP), alkaline phosphatase (AP), and beta-galactosidase (beta Gal).

Expression of the NotchΔE can be driven by a constitutive promoter, e.g., SV40, CMV, UBC, EF1A, PGK, or CAGG (Qin et al., PLoS One. 2010; 5(5): e10611).

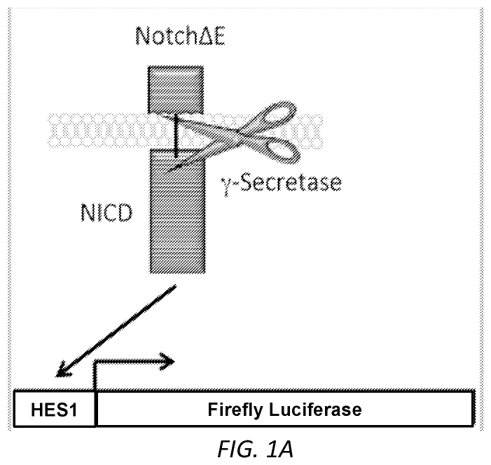

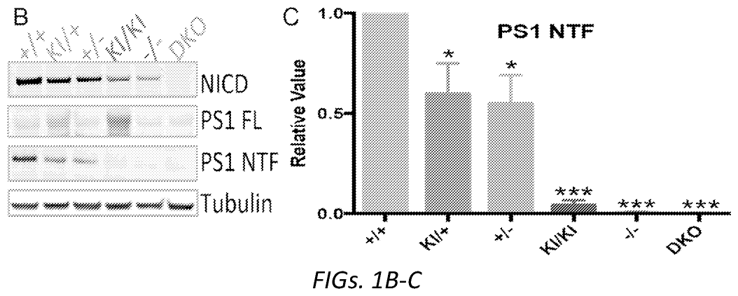

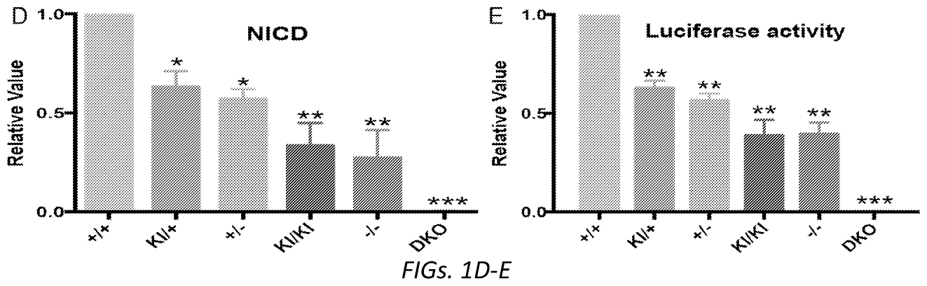

The assay is preferably performed in living cells, e.g., mammalian cells, e.g., cultured mammalian cells. Exemplary cell lines include CHO, Hela, COS 7, COPS or HEK (293 or 293T) cells; other cells such as mouse or human embryonic fibroblasts (MEFs) can also be used. The cells can stably express one or both of the reporter gene and NotchΔE, or can transiently express one or both. In some embodiments, two plasmids are used. Each plasmid can include a different antibiotic resistance gene, e.g., kanamycin; spectinomycin; streptomycin; ampicillin; carbenicillin; bleomycin; erythromycin; polymyxin b; tetracycline; or chloramphenicol resistance genes. In some embodiments, the reporter gene is in a hygromycin resistant plasmid (e.g., Hes1-Luc-Hygro) and the NotchΔE is in a neomycin resistant plasmid (CMV-NΔE-Neo) (e.g., as shown in FIG. 1A).

In some embodiments, the cells include one or more mutations in an endogenous gene that is associated with Alzheimer's disease, e.g., with familial Alzheimer's disease (FAD), e.g., mutations in the APP, PSEN1, and/or PSEN2 genes encoding beta amyloid protein precursor (APP), presenilin-1 (PS1) and Presenilin2 (PS2). See, e.g., Lanoiselee et al., PLoS Med. 2017 March; 14(3): e1002270 and PCT/US2019/033616.

Methods of Screening

Included herein are methods for screening test compounds, e.g., polypeptides, polynucleotides, inorganic or organic large or small molecule test compounds, to identify agents useful as gamma secretase agonists, e.g., useful in the treatment of disorders associated with loss of or reduced gamma secretase activity, e.g., disorders that are suitable for treatment with gamma secretase agonists. Such disorders can include Alzheimer's disease (Xia et al., Neuron. 2015 Mar. 4; 85(5):967-81).

As used herein, “small molecules” refers to small organic or inorganic molecules of molecular weight below about 3,000 Daltons. In general, small molecules useful for the present methods have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods described herein can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In the present methods a test compound is applied to a cell comprising an assay system as described herein and the effect of the test compound on expression of the reporter gene is evaluated. An increase in signal from the reporter gene (e.g., an increase in fluorescence or luminescence) indicates that the test compound is a gamma secretase activator.

Methods for detecting increased fluorescence are known in the art and include high-throughput imaging (HTI) methods that use automated microscopy and image analysis (see, e.g., Pegoraro and Misteli, Trends Genet. 2017 September; 33(9): 604-615) to screen large numbers of test compounds substantially simultaneously.

A test compound that has been screened by a method described herein and determined to be a gamma secretase activator can be considered a candidate compound. A candidate compound that has been screened, e.g., in an in vivo model of a disorder, e.g., Alzheimer's disease, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that are identified as gamma secretase activators) in a first screen can be selected and systematically altered, e.g., using rational design, to optimize binding affinity, avidity, specificity, or other parameter. Such optimization can also be screened for using the methods described herein. Thus, in one embodiment, the methods include screening a first library of compounds using a method described herein, identifying one or more hits in that library, subjecting those hits to systematic structural alteration to create a second library of compounds structurally related to the hit, and screening the second library using the methods described herein. The methods can also include screening hits identified using the present methods using a second methods, e.g., an assay that detects processing of Notch and/or APP, e.g., as described herein.

Test compounds identified as hits can be considered candidate therapeutic compounds, useful in treating disorders as described herein, e.g., Alzheimer's disease. A variety of techniques useful for determining the structures of “hits” can be used in the methods described herein, e.g., NMR, mass spectrometry, gas chromatography equipped with electron capture detectors, fluorescence and absorption spectroscopy. Thus, the methods can also include compounds identified as “hits” by the methods described herein, and methods for their administration and use in the treatment, prevention, or delay of development or progression of a disorder described herein.

Test compounds identified as candidate therapeutic compounds can be further screened by administration to an animal model, e.g., of Alzheimer's disease (see, e.g., Vitek et al., Alzheimers Dement (N Y) 2020; 6(1): e12114). The animal can be monitored for a change in the disorder, e.g., for an improvement in a parameter of the disorder, e.g., a parameter related to clinical outcome. In some embodiments, the parameter is production of amyloid plaques and neurofibrillary tangles, and an improvement would be a reduction in levels or rate of production of amyloid plaques and neurofibrillary tangles. In some embodiments, the subject is a human, e.g., a human with Alzheimer's disease, and the parameter is cognitive impairment.

### EXAMPLES

The invention is further described in the following examples, which do not limit the scope of the invention described in the claims.

Methods

The following materials and methods were used in the Examples below.

Plasmids:

A 378-bp HindIII-KpnI fragment of Hes, which contains the region from −194 to +160 of the mouse Hes1 promoter (Watanabe et al., J Biol Chem. 2009 May 15;284(20):13705-13713) was cloned into the multicloning site of pGL4.15 (promega) to generate Hes1-luc-hygro. To create CMV-NAE-Neo, a 1,863-bp NHeI-NHeI fragment of notchΔE-cMyc (Kopan et al., Proc Natl Acad Sci USA. 1996 Feb. 20; 93(4):1683-8) was cloned into the multiple cloning site of pC1-CMV-Neo (Promega).

Cell Lines and Transient Transfection:

Mouse embryonic fibroblasts (MEFs) with different PS1 genotype (+/+, L435F KI/+, +/−, L435F KI/KI, −/−, and DKO) were maintained in media supplemented with 10% FBS and 1% penicillin and streptomycin. MEFs were immortalized via transfection with 1 μg SV40 and passaged against GFP transfected cells till immortalization is confirmed. MEFs were also transfected with 1.25 μg Hes1-luc, 5 ng CMV-NotchΔE, and an increasing amount of plasmid expressing human PS1 in 6-well plates.

Screening Compounds:

For screening, cells were seeded in 384-well plates using Thermo Multidrop Combi at a density of 2,500 cells in 30 μl media and compounds were transferred by Seiko Compound Transfer Robot on the same day. For dose response, compounds were dispensed via the Hewlett Packard D300.

Western Analysis:

24 hours after transfection, cells were lysed in RIPA buffer (50 mM Tris-Cl (pH 7.6), 150 mM NaCl, 1% NP40, 0.5% sodium deoxycholate, protease inhibitor cocktail (Sigma), 1 mM DTT). Proteins were separated in NuPAGE gels (Invitrogen) and transferred to nitrocellulose membranes. Primary antibodies were rabbit anti-cleaved notch1 val1744 (cell signaling), rabbit anti-PS1 NTF (Calbiochem), and rabbit anti-tubulin (cell signaling). Membranes were then incubated with dye-coupled secondary antibodies (goat anti-rabbit IRdye800, and goat anti-rabbit IRdye680 from Licor). Signals were quantified with the Odyssey Infrared Imaging System (LI-COR Bioscience).

Luciferase Assay:

The steady glo luciferase assay system (Promega) was used to determine firefly luciferase activity per manufacturer's instructions. Luciferase activity was detected 24 hours after transfection using a plate reader (Perkin Elmer EnVision).

Quantitative Reverse Transcriptase-PCR (Q-RT-PCR):

RNA was extracted and treated with DNase I per manufacturer's instructions (Zymo Research). The RNA was then reverse transcribed (Bio-Rad) in the presence of gene specific primers. The primers used in the RT reaction are 5′-CATTGTCATACCAGGAAATGAG-3′ (SEQ ID NO:1) for GAPDH, 5′CTCGAAGCATTAACCCTCAC-3′ (SEQ ID NO:2) for notchΔE, and 5′-GTGAGAATTCACGGCGATCT-3′ (SEQ ID NO:3) for firefly luciferase. PCR reactions were performed using SYBR green PCR master mix in 7500 Fast Real-Time PCR system (Applied Biosystems) with cDNA and gene specific primer pairs. Reactions were performed in triplicate, and threshold cycle values were normalized to GAPDH. The primer pairs used in this study are as follows: 5′-GGGTGTGAACCACGAGAAAT-3′(SEQ ID NO:4) and 5′-CCTTCCACAATGCCAAAGTT-3′ (SEQ ID NO:5) for GAPDH, 5′-AGCTTTTGCTCCATAGCTTTA-3′ (SEQ ID NO:6) and 5′-AAGGAAGCTAAGGACCTCAA-3′ (SEQ ID NO:7) for notchΔE, and 5′-GGCTGCAAAAGATCCTCAAC-3′ (SEQ ID NO:8) and 5′-AATGGGAAGTCACGAAGGTG-3′(SEQ ID NO:9) for luciferase.

Stable Cells:

MEFs (1.5×10⁶) were seeded in two 10-cm tissue culture plates; one to be used as a negative control. The next day, one plate is transfected with 9 μg CMV-NotchΔE (neomycin) and 7.511 g Hes1Luciferase (hygromycin) plasmids. 24-hours later, cells in both plates were passaged 1:100, 1:200 and 1:1000 and maintained in DMEM media supplied with 10% FBS, 100 ug/ml Hygromycin, and 200 μg/ml G418. Media was changed every 3 days and cells were checked daily ensuring that cells in the untransfected plates (negative control) have died. Within two weeks, hygromycin/G418 resistant colonies were formed with a diameter of approximately 5 mm. Each clone was transferred into a separate well of a 24-well plate. When confluent, MEFs were transferred into 6-well plates then into 10 cm dishes. For each clone, some cells were frozen while others harvested to test luciferase and notchΔE expression via luciferase assay, western blot and Q-RT-PCR.

### Example 1. Genotypic Effect of PS on γ-Secretase Activity

For the identification of compounds that could increase the activity of γ-secretase, a Notch target gene reporter system was created. It contains two plasmids: Hes1 proximal promoter containing two Notch responsive CBF1/RBPJκ binding sites driving expression of a firefly luciferase in a hygromycin resistant plasmid (Hes1-Luc-Hygro), and CMV promoter and NotchΔE cloned in a neomycin resistant plasmid (CMV-NΔE-Neo) (FIG. 1A).

To determine the impact of FAD mutations on γ-secretase activity, we generated MEFs expressing FAD-linked mutation that were extracted from our PS1 L435F KI and KO mice. MEFs with varying PS1 genotypes (+/+, L435F KI/+, +/−, L435F KI/KI, −/−, and DKO) transfected with NotchΔE were used to measure γ-secretase activity. The western blot analysis shows that NICD cleavage is dependent on γ-secretase activity, which is determined by PS1 genotype (FIG. 1B-D). The same MEFs cotransfected with Hes1-Luc were used to measure luciferase activity pending the liberation of Hes-1 promoter from co-repressor proteins and the recruitment of co-activators (FIG. 1A). Similar to the western blot data, the luciferase activity reflects NICD cleavage (FIG. 1E).

### Example 2. Rescue of γ-Secretase Impairment by hPS1

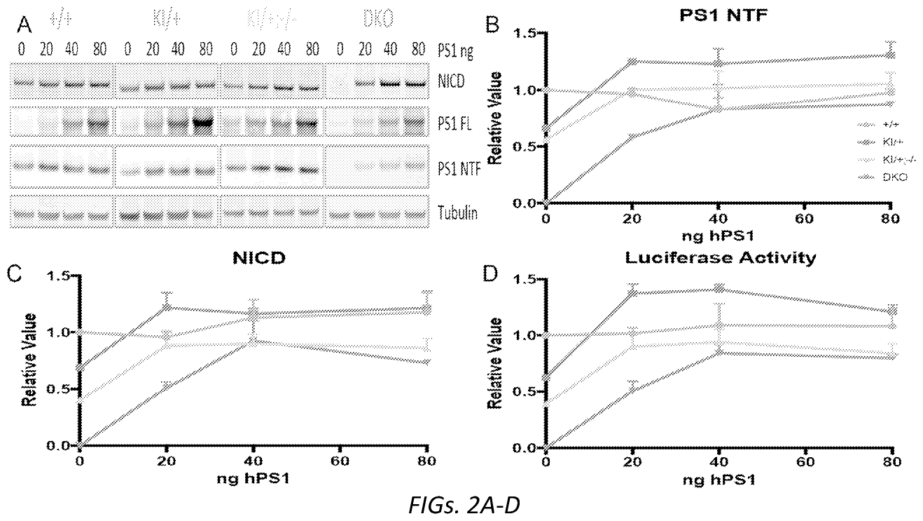

To determine whether the reduction of luciferase activity from the Hes1-Luc reporter in PS KI and KO MEFs is intrinsic to the loss of PS, we next examined whether introduction of exogenous human PS1 (hPS1) can rescue this transcriptional defect. For this purpose, we transfected varying amount of wild-type hPS1 cDNAs into the different PS1 genotype MEFs. The presence of human PS1 was confirmed by Western blotting (FIG. 1A-B). Introducing exogenous hPS1 was able to increase NICD cleavage in PS1 KI and KO MEFs (FIGS. 2A, C). These results were also confirmed when luciferase activity was measured (FIG. 2D). Interestingly, wild-type MEFs showed no increase in γ-secretase activity when transfected with exogenous hPS1. These results indicate that exogenous hPS1 can rescue the impaired γ-secretase activity in PS1 KI and KO MEFs.

### Example 3. Development of Cell Based Assay for Screening

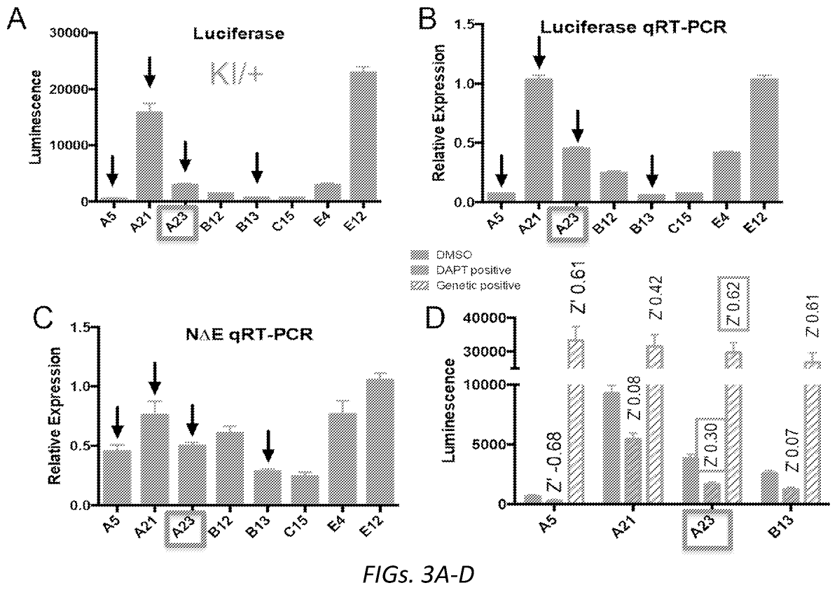

For the identification of compounds that could increase γ-secretase activity, L435F KI/+ stable cells expressing both CMV-NΔE-Neo and Hes1-Luc-hygro were generated. Luciferase expression in the selected clones was confirmed via a luciferase assay (FIG. 3A). We also performed quantitative RTPCR using primers specific for luciferase and Notch-cMyc to confirm their expression (FIG. 3B-C).

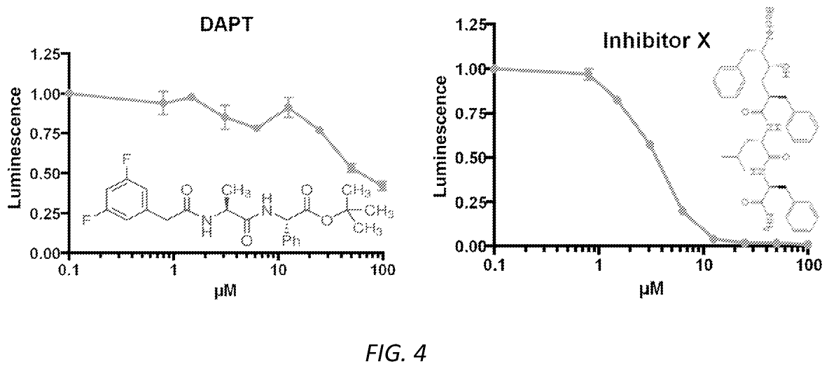

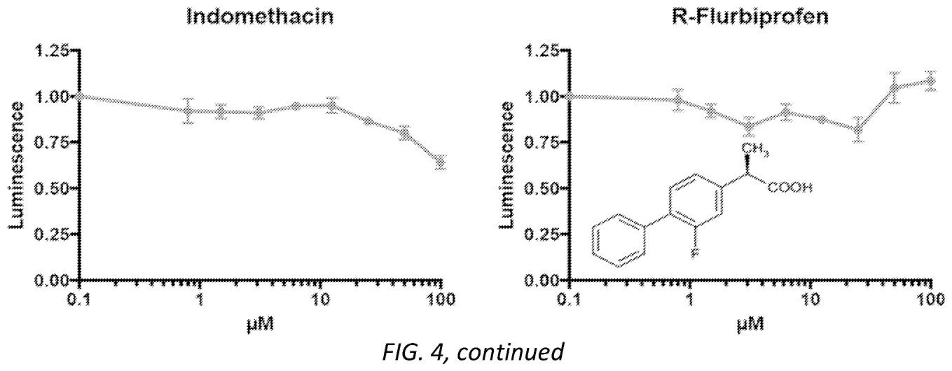

After generating the stable cells, 384-well plates were optimized in the EnVision plate reader for plate dimension, and crosstalk correction. To provide a robust (high Z′ factor) HTS-compatible assay, four clones expressing different levels of NotchΔE and luciferase (FIG. 3A-C, black arrows) were compared to identify the maximum difference in signal between DMSO, and DAPT (γ-secretase inhibitor), as well as genetic positive (a clone expressing high levels of luciferase) controls. For each clone seeded in the 384-well plate, 100 nl DMSO was transferred to ⅓ of the plate, while 100 nl DAPT (30 mM) was added to another ⅓ of the plate. The last ⅓ of the plate was seeded with a genetic positive control (a clone expressing high levels of luciferase). Clone A23 with medium luciferase activity (compared to other clones) showed the best Z′ factor (FIG. 3D, green box). Clone A23 was further validated by testing the effect of γ-secretase inhibitors (DAPT and L685,458), and modulators (Indomethacin and R-Flurbiprofen). The reduction of luciferase activity was confirmed in the γ-secretase inhibitors with lesser effect from the γ-secretase modulators (FIG. 4).

### Example 4. High Throughput Screening of Known Bioactive and Commercial Library

To evaluate the reproducibility of the assay in an HTS-format, we screened 7 plates containing 1,902 compounds of the Selleck bioactive compound library (ICCB-Longwood) at 30 μM under optimal assay conditions in duplicate. Each screening plate included the genetic positive control for identifying plate-to-plate variability and establishing assay background levels. Average, median and standard deviation were calculated for all experimental wells on each assay plate. We generated graphs to visualize quality control parameters utilizing Dotmatics Vortex software. We checked the luminescent values in the duplicate plate to confirm a good correlation of replicates. A heat map and a 3D histogram were also generated for each plate, which showed no noticeable patterns or edge effect. The data was also normally distributed. These observations demonstrate that the assay is highly reliable, reproducible, and well suited for HTS screening.

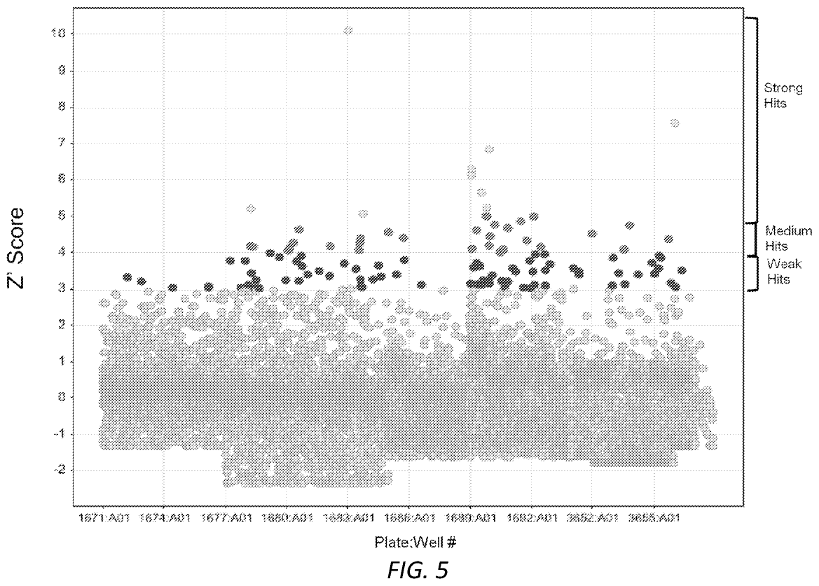

After confirmation of the performance of the assay, 8,098 compounds from Asinex commercial library (ICCB-Longwood) were screened at 10 μM. The average and standard deviations were then used for experimental well Z′ score calculation. The raw data distribution was converted to a standard normal distribution with a mean of 0 and a standard deviation of 1. Each Z′ score for a well replicate represented the number of standard deviations above the plate mean of the experimental wells. Hits were then categorized according to the Z′ score (3-4 weak, 4-5 Medium, >5 strong). Wells were considered positive if both replicates were scored as strong, medium or weak. With replicates, we only considered the lower Z′ score. From the total 10,000 compounds screened 92 confirmed hits were identified (FIG. 5). Three of these were shown to be unstable at the screening facility. Thus, the final hit rate was 0.89%.

### Example 5. Confirmation and Validation of Hits

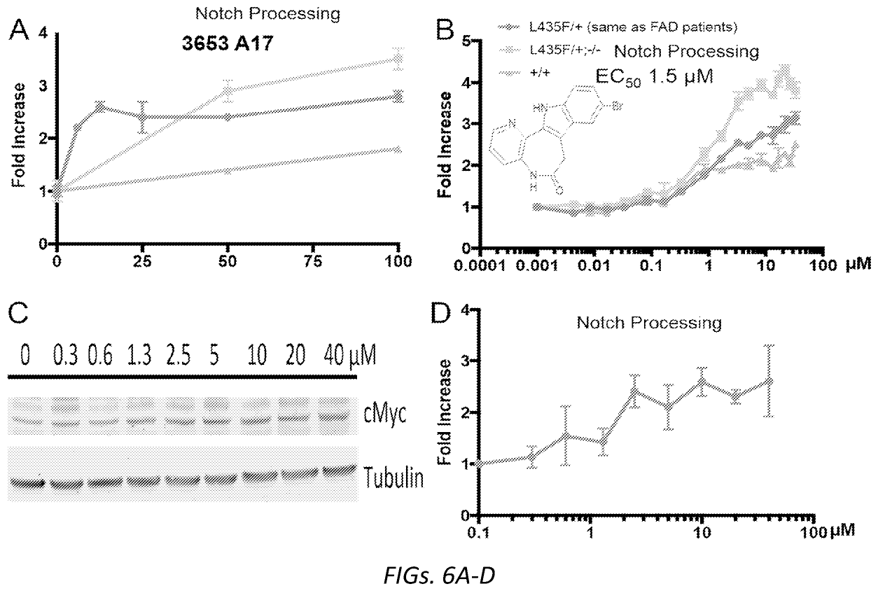

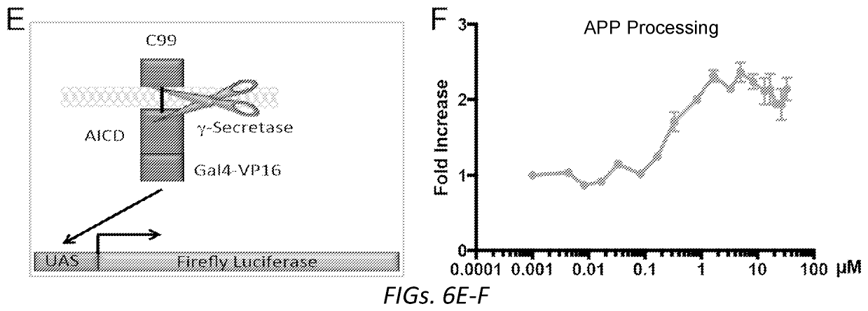

FIG. 6 shows a selected hit (3653 A17) as an example for the confirmation process carried out for all hits. Small aliquots of the hits from the same library were used to confirm the effect on the same cells (PS1 KI/+) as well as stable cells with different genotypes; wild-type cells and PS1 KI/+;PS2 −/− cells (FIG. 6A). Compounds showing higher fold increase in the KI cells compared to the wild-type cells were given priority, purchased and reconstituted in DMSO for further validation. This allowed confirming if another patch of the same compound would produce the same phenotype. Dose response curves (4 nM to 33 μM) were generated allowing for EC₅₀ calculations (FIG. 6B). The increase of Notch cleavage by these compounds was also confirmed by Western blot (FIG. 6C-D).

To identify compounds that rescue two independent and well-established functional measures of γ-secretase activity (processing of Notch and APP), we utilized an APP target gene reporter system as a secondary screen. It contains two plasmids: a Gal4-responsive firefly luciferase reporter in a hygromycin resistant plasmid (UAS-Luc-Hygro), and CMV promoter and C99 (a C-terminal derivative of APP) fused to the transcriptional activator Gal4-VP16 cloned in a neomycin resistant plasmid (CMV-C99-Gal4VP16-Neo). The luciferase activity in this reporter is dependent on the activation of the UAS promoter after the cleavage of C99-Gal4-VP16 (FIG. 6E). L435F KI/+ stable cells expressing both UAS-LucHygro and CMV-C99-Gal4-VP16-Neo were generated. Hits were confirmed in the secondary assay and dose response curve was generated (FIG. 6F).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated cell comprising:
(i) a reporter gene under control of a promoter comprising at least one CBF1 binding site, and
(ii) a sequence encoding NotchΔE under control of a promoter, preferably wherein the cell comprises one or more mutations in an endogenous APP, PSENJ, and/or PSEN2 gene, preferably wherein the mutations are associated with Alzheimer's disease.

2. The isolated cell of claim 1, wherein the promoter comprising at least one CBF1 binding site is a Hes1 proximal promoter.

3. The isolated cell of claim 1, wherein the sequence encoding Notch11E is under control of a constitutive promoter.

4. The isolated cell of claim 3, wherein the constitutive promoter is SV40, CMV, UBC, EF1A, PGK, or CAGG.

5. The isolated cell of claim 1, wherein the reporter gene encodes a fluorescent protein.

6. The isolated cell of claim 5, wherein the fluorescent protein is a green fluorescent protein, a red fluorescent protein, a yellow fluorescent protein, a blue fluorescent protein, a cyan fluorescent protein, or an orange fluorescent protein.

7. The isolated cell of claim 1, wherein the reporter gene encodes a bioluminescent protein.

8. The isolated cell of claim 7, wherein the bioluminescent protein is aequorin or luciferase.

9. The isolated cell of claim 1, which stably expresses the reporter gene and/or NotchLΔE.

10. The isolated cell of claim 1, which transiently expresses the reporter gene and/or NotchLΔE.

11. The isolated cell of claim 1, which is a mammalian cell, preferably a cultured mammalian cell.

12. A method of identifying a gamma secretase activator, the method comprising providing a cell comprising:
(iii) a reporter gene under control of a promoter comprising at least one CBF1 binding site, and
(iv) a sequence encoding Notch11E under control of a promoter;
preferably wherein the cell comprises one or more mutations in an endogenous APP, PSENJ, and/or PSEN2 gene, and preferably wherein the mutations are associated with Alzheimer's disease;
contacting the cell with a test compound;
detecting a signal from the reporter gene in the presence and absence of the test compound;
comparing the signal from the reporter gene in the presence and absence of the test compound; and
identifying as a gamma secretase activator a compound that produces an increased signal from the reporter gene in the presence of the test compound as compared to the signal in the absence of the test compound.

13. The method of claim 12, wherein the promoter comprising at least one CBF1 binding site is a Hes1 proximal promoter.

14. The method of claim 12, wherein the sequence encoding Notch1AE is under control of a constitutive promoter.

15. The method of claim 14, wherein the constitutive promoter is SV40, CMV, UBC, EF1A, PGK, or CAGG.

16. The method of claim 12, wherein the reporter gene encodes a fluorescent protein.

17. The method of claim 16, wherein the fluorescent protein is a green fluorescent protein, a red fluorescent protein, a yellow fluorescent protein, a blue fluorescent protein, a cyan fluorescent protein, or an orange fluorescent protein.

18. The method of claim 12, wherein the reporter gene encodes a bioluminescent protein.

19. The method of claim 18, wherein the bioluminescent protein is aequorin.

20. The method of claim 18, wherein the bioluminescent protein is luciferase, and the method further comprises lysing the cell and contacting the lysate with a luciferin substrate.

21. The method of claim 12, wherein the cell stably expresses the reporter gene and/or NotchLΔE.

22. The method of claim 12, wherein the cell transiently expresses the reporter gene and/or NotchLΔE.

23. The method of claim 12, wherein the cell is a mammalian cell, preferably a cultured mammalian cell.

24. The method of claim 12, wherein detecting a signal from the reporter gene comprises using imaging to detect a fluorescent or bioluminescent signal.
